(19) United States
(12) Patent Application Publication
(10) Pub. No.: US 2026/0060616 A1
(43) Pub. Date: Mar. 5, 2026

(54) CAPSULE SENSOR STRUCTURE AND ASSEMBLY METHOD THEREOF

(71) Applicant: DotSpace Inc., Wilmington, DE (US)

(72) Inventors: Uei-Ming Jow, San Diego, CA (US); Yen-Liang Lai, Taipei City (TW)

(21) Appl. No.: 18/822,487

(22) Filed: Sep. 3, 2024

(51) Int. Cl. A61B 5/00; A61B 5/07

(52) U.S. Cl. CPC A61B 5/6861; A61B 5/07; A61B 2562/12; A61B 2562/162

(57) ABSTRACT

A capsule sensor structure includes a housing, an assembly frame, an energy module, an actuation module and a control module, the housing is a capsule body including an accommodation slot and a cap, and the accommodation slot is assembled to the cap to form a seal structure. The assembly frame contains the energy module, and the actuation module and the control module are arranged at the top and bottom of the assembly frame respectively. The energy module is telecommunicatively connected to the actuation module and the control module, the control module is telecommunicatively connected to the actuation module and an external monitoring system. While the capsule sensor structure is put into a living organism, the control module receives an instruction of the monitoring system to drive the actuation module to perform biosignal detection or physical therapy and give an operation result to the monitoring system for remote monitoring.

### TECHNICAL FIELD

The present disclosure relates to a signal sensor, and more particularly relates to a capsule sensor structure and its assembly method.

### BACKGROUND OF THE DISCLOSURE

In order to overcome the difficulties in medical treatment, a digital medical treatment using advanced packaging technology to integrate the mini PCBA circuit board, micro sensor, antenna and battery into a single-packaged precision structure of a capsule body becomes increasingly eye-catching, which allows patients to swallow the capsule sensor as if they were taking medicine and then through pressure, image, ultrasound, or gas sensors to sense the vital signs and transmit the vital signs through wireless transmission via an antenna to an external monitoring system for diagnosis and treatment. In addition, with the development of nano-size integrated circuits, the mechanical switches used in the circuits of the capsule sensor have been replaced with integrated circuit (IC) switches, resulting in the improvements of smaller size and lower cost. Yet, a drawback that the IC switches still have a very small operating current when they are turned off. In other words, even if the capsule sensor is turned off, it is still not at a real power-off status, but it is at a standby status operating at a very low current state. If a small current leakage is generated by the rest of the circuit components, it will greatly shorten the shelf life of the capsule sensor, which is not conducive to the storage of medical devices.

Furthermore, present capsule sensors use adhesives such as epoxy resin in the assembly and packaging process, but such adhesives have small conductivity in the liquid state, resulting in a loss of battery power and a leakage of current in the circuit components due to the spillage of the adhesives into the circuit components during the adhesive dispensing process, which in turn causes damages to the whole capsule sensor, shortening the shelf life or lowering the stability of the capsule sensors. In view of the aforementioned drawbacks, the present discloser attempt to find a way to overcome these drawbacks by improving the related-art capsule sensor structure to avoid the leakage of current caused by the overflow or spillage of adhesives during the adhesive dispensing and packaging processes, and this is the subject of the present disclosure.

### SUMMARY OF THE DISCLOSURE

The primary objective of the present disclosure is to provide a biosignal sensor with a high waterproof property and a function of preventing current leakage in liquid adhesives during the manufacturing process. The biosignal sensor will provide sufficient shelf life, correct operation, and high sensing precision while being putting into a living organism for biological signal detection or for physical therapy.

To achieve the aforementioned objective, the present disclosure discloses a capsule sensor structure telecommunicatively connected to an external monitoring system and including: a housing, substantially in the shape of a capsule body and having an accommodation slot and a cap, the accommodation slot being dispensed with an adhesive and bonded with the cap to form a seal structure; an assembly frame, installed in the seal structure; an energy module, installed in the assembly frame; an actuation module, installed at the top of the assembly frame and electrically connected to the energy module; and a control module, installed at the bottom of the assembly frame, electrically connected to the energy module, telecommunicatively connected to the actuation module, and provided with an antenna which is used for telecommunicatively connecting to the monitoring system. While the capsule sensor structure is put into a living organism, the control module receives an instruction of the monitoring system to drive the actuation module to perform a biosignal detection or a physical therapy, feeds back an operation result and transmits the operation result to the monitoring system for remote monitoring.

The capsule sensor structure further includes an assembly part for connecting the assembly frame to securely install the energy module, the actuation module and the control module in the assembly frame, the assembly frame is substantially in a cylindrical shape and sequentially from top to bottom has a first compartment, a second compartment and a third compartment, the first compartment is provided for accommodating a circuit board of the actuation module, the second compartment is provided for accommodating a battery and a metal electrode sheet of the energy module, and the third compartment is provided for accommodating a circuit board of the control module; the antenna is wrapped onto a sidewall of the assembly frame, and the opposite sidewall of the assembly frame is a hollow sidewall provided for placing the assembly part, with the whole assembly placed into a sealed structure after assembling.

The assembly part includes a first part, the left and right sides of the first part extend in an arc forward to form a second part and a third part respectively, and both the area of the second part and the area of the third part are smaller than the area of the first part, the upper side of the first part extends vertically forward to form a fourth part, and the front end of the fourth part is provided with a first concave arc; the top of a side of the assembly frame correspondingly assembled with the assembly part is configured to be corresponsive to the fourth part of the assembly part with a concavely designed assembly slot, and the front end of the assembly slot is configured to be corresponsive to the first concave arc and provided with a second concave arc, such that while the assembly part is assembled to the assembly frame and the fourth part is assembled to the assembly slot, the first concave arc is coupled to the second concave arc to form a round hole for accommodating an actuating element of the actuation module.

In addition, the actuation module and the control module have part or all of the circuit components coated with an insulating material to form an insulating layer to improve the overall structural insulation. After the round hole accommodates the actuating element, the periphery of the round hole is dispensed with an adhesive to form a first sealing ring to improve the waterproof property. The inner side of the open end of the accommodation slot is surrounded with a glue overflow groove for accommodating excessive adhesive materials after the accommodation slot and the cap are dispensed with an adhesive and bonded with each other. The capsule sensor structure further includes an O-ring installed to the cap and clamped between the cap and the accommodation slot to improve the sealing strength between the cap and the accommodation slot to further perfect the structural waterproof property. The control module includes a contactless switch.

The capsule sensor structure further includes a filter membrane wrapped on the actuation module to prevent the actuating element from being damaged by the penetration of a biological tissue fluid or moisture. The filter membrane and an opening of the accommodation slot are dispensed with an adhesive and bonded with each other to improve the structural assembly sealing strength and perfect the structural waterproof property. The top side of the assembly frame assembled with the assembly part is concavely formed with at least one dispensing groove for preventing the adhesive material from overflowing and contaminating the actuation module while dispensing and adhering the filter membrane to the top side of the assembly frame assembled with the assembly part. The filter membrane is substantially in a donut shape, an opening is formed at the center of a round slice and provided for accommodating the actuating element, and the filter membrane is attached onto the actuation module and the periphery of the filter membrane is coupled to the opening above the accommodation slot; the periphery of the opening is dispensed with an adhesive to form a second sealing ring to improve the waterproof property. The housing is formed by a polycarbonate (PC) containing biocompatible material, and the assembly frame and the assembly part are made of a polyamide (PA) containing non-biocompatible material. The assembly frame and the assembly part is made of polyamide (PA) and a hardness strengthening material. The hardness strengthening material comprises a fiber material which accounts for 10-40% of the total production materials.

To achieve another objective, the present disclosure further discloses a method of assembling the aforementioned capsule sensor structure, which includes the steps of: covering a silicone protective cover onto an actuating element of the actuation module; completely immersing the circuit board of the actuation module in an insulating material, and setting it still in the adhering insulating material to solidify to form an insulating layer that covers the surface of the circuit board of the actuation module, and then removing the silicone protective cover; assembling the circuit board of the actuation module to the upper part of the assembly frame, assembling the circuit board of the control module to the lower part of the assembly frame, surrounding the antenna around a sidewall of the assembly frame, and assembling the energy module into the assembly frame; assembling the assembly part to the assembly frame to form a semi-assembled product; placing the semi-assembled product into the accommodation slot; and dispensing and assembling the cap and the accommodation slot.

In summation, the present disclosure based on the following technical characteristics enables the overall structure to exhibit the advantages of high sealing strength, high waterproof resistance, and effective prevention of current leakage, thus ensuring the high shelf life, high accuracy of operation, and high quality constancy of the capsule sensor structure:
(1) By the setup of the hollow side of the assembly frame and the first compartment, the second compartment and the third compartment in the assembly frame, the metal electrode sheet of the energy module can be separated from the upper and lower circuit components to minimize damages to the circuit components in the event of current leakage.
(2) By the setup of the first concave arc of the assembly part and the second concave arc of the assembly frame to form the round hole, it allows the actuating element to protrude out of the top surface of the assembly frame assembled with the assembly part and separate the actuating element from the lower circuit component, thus further preventing the circuit components from being easily affected, damaged, or short-circuited by moisture.
(3) By dispensing adhesive to the filter membrane at the opening, dispensing adhesive between the filter membrane and the opening of the accommodation slot, dispensing adhesive to the round hole on the assembly frame, dispensing adhesive between the accommodation slot and the cap, or even dispensing adhesive along the assembly track of the assembly part and the assembly frame, and dispensing adhesive between the assembly frame and the opening of the accommodation slot, the adhesive material can be used effectively to form a sealing ring or sealing strip, which can greatly improve the waterproof property of the overall structure.
(4) By the setup of the O-ring, the structural sealing strength of the housing can be enhanced.
(5) By coating an insulating material to form the waterproof protective membrane on the circuit components, it can prevent the leakage of current from shortening the shelf life of the circuit components, due to the liquid sealing adhesive material flowing down in the glue dispensing and assembly processes.
(6) By the setup of the assembly frame, the assembly parts, and the housing, the sealing direction of the overall structure is situated at the sensor, that is, around the actuating element rather than at the sidewall of the capsule sensor, thus minimizing the trouble of the tailored structure of the side of the assembly, and increasing the occlusal force between the structural components to enhance the bonding strength of the overall structure, so as to effectively prevent loosening circuit components and lowering the operation quality of the sensor.
(7) By the design of the glue overflow groove and the dispensing groove, the adhesive materials can be buffered during the glue dispensing process, furthermore, the structural characteristics of the glue overflow groove can be used to prevent excessive adhesive materials from spilling and contaminating the circuit components so as to effectively prevent the components from being damaged by the leakage of current and ensuring the shelf life and quality of use of the capsule sensor structure.

### BRIEF DESCRIPTION OF THE DRAWINGS

FIG. 1A is a partial disassembled diagram of a capsule sensor structure of a first preferred embodiment of the present disclosure;

FIG. 1B is an exploded view of a capsule sensor structure of the first preferred embodiment of the present disclosure;

FIG. 2A is a partial disassembled diagram of a capsule sensor structure of a second preferred embodiment of the present disclosure;

FIG. 2B is an exploded view of a capsule sensor structure of the second preferred embodiment of the present disclosure;

FIG. 3 is a schematic diagram of a partial assembly of a capsule sensor structure of the second preferred embodiment of the present disclosure;

FIG. 4 is a cross-sectional diagram of a partial assembly of a capsule sensor structure of the second preferred embodiment of the present disclosure;

FIG. 5 is a cross-sectional diagram of a capsule sensor structure of the second preferred embodiment of the present disclosure;

FIG. 6 is a partial disassembled diagram of a capsule sensor structure of a third preferred embodiment of the present disclosure; and

FIG. 7 is a schematic diagram showing the assembly procedure of a capsule sensor structure of a fourth preferred embodiment of the present disclosure.

### DETAILED DESCRIPTION OF THE PREFERRED EMBODIMENTS

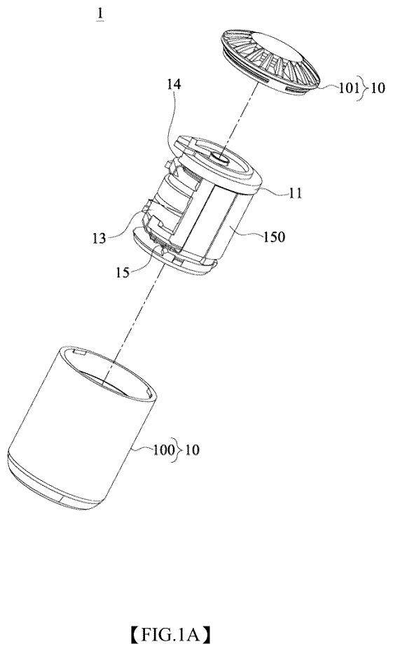

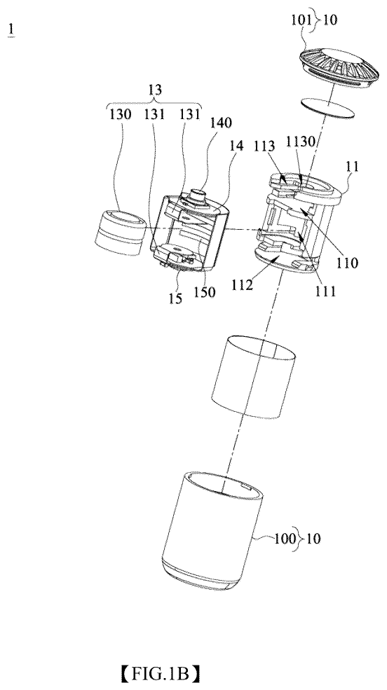

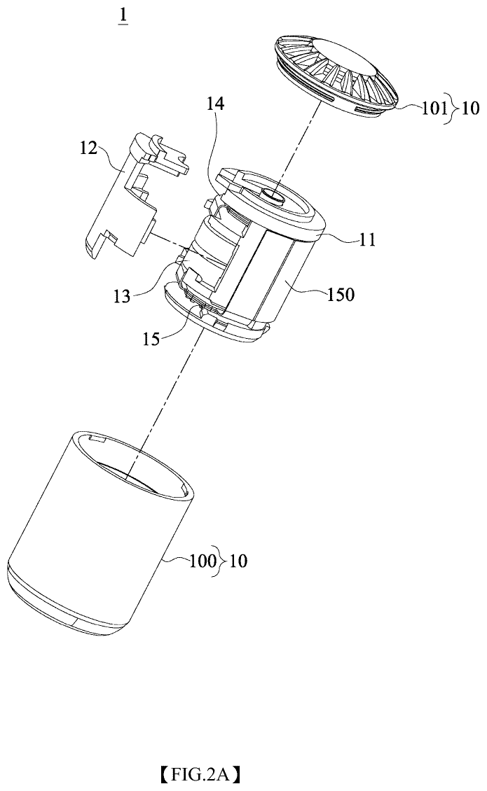

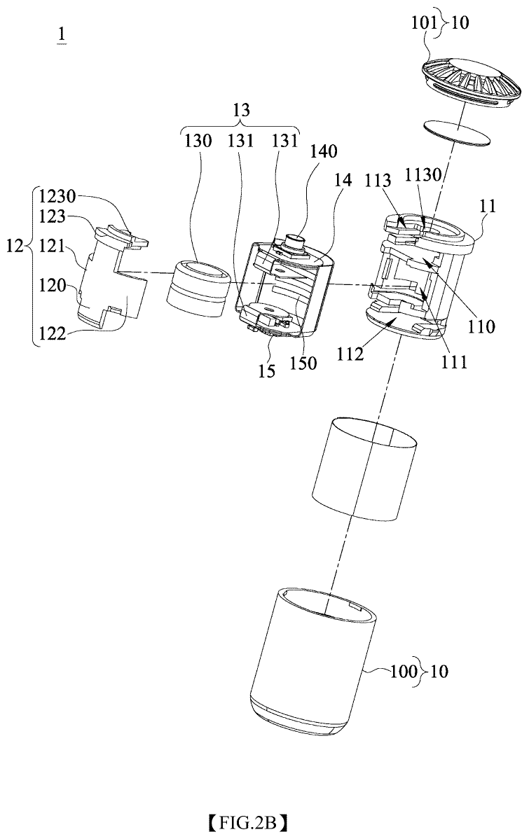

With reference to FIGS. 1A and 2A for the partial disassembled diagrams of a capsule sensor structure in accordance with the first and second preferred embodiments of the present disclosure respectively, the capsule sensor structure 1 is telecommunicatively connected to an external monitoring system (not shown in the figure) and includes a housing 10, an assembly frame 11, an assembly part 12, an energy module 13, an actuation module 14 and a control module 15. The housing 10 is in the shape of a capsule body and provided with an accommodation slot 100 and a cap 101, and the accommodation slot 100 is dispensed with an adhesive and assembled with the cap 101 to form a seal structure. The energy module 13 is installed in the assembly frame 11 and electrically connected to the actuation module 14 and the control module 15, the actuation module 14 is installed at the upper part of the assembly frame 11 and telecommunicatively connected to the control module 15, the control module 15 is installed at the lower part of the assembly frame 11, provided with an antenna 150, and telecommunicatively connected to the monitoring system through the antenna 150. The assembly part 12 is provided to be assembled with the assembly frame 11 in order to securely install the energy module 13, the actuation module 14 and the control module 15 into the assembly frame 11 and then into the seal structure. While the capsule sensor structure 1 is put into a living organism, the control module 15 receives an instruction of the monitoring system to drive the actuation module 14 to perform a biosignal detection or physical therapy, and a feedback of an operation result, and the control module 15 transmits the operation result to the monitoring system for remote monitoring.

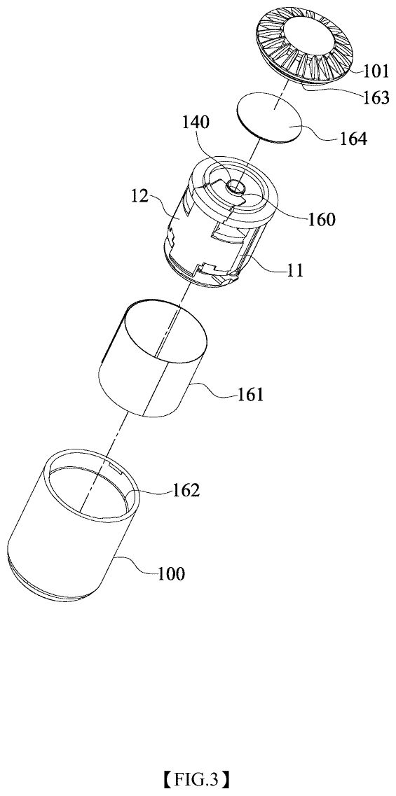

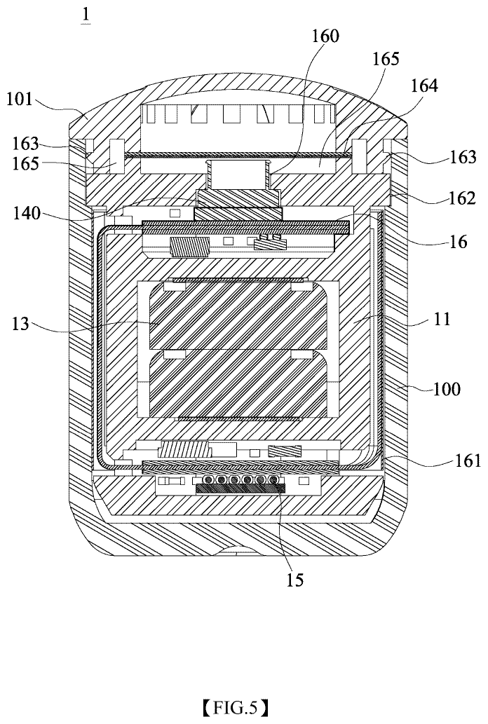

With reference to FIGS. 1B, 2B-5, and 6 for the exploded views, schematic diagrams and cross-sectional diagrams of the first and second preferred embodiments and the disassembled diagram of the third preferred embodiment of the present disclosure respectively, the capsule sensor structure 1 is telecommunicatively connected to an external monitoring system (not shown in the figure) and contains a housing 10, an assembly frame 11, an assembly part 12, an energy module 13, an actuation module 14 and a control module 15. The housing 10 is in the shape of a capsule body and provided with an accommodation slot 100 and a cap 101, and the accommodation slot 100 is dispensed with an adhesive and assembled with the cap 101 to form a seal structure. The assembly frame 11 is in a substantially cylindrical shape and from top to bottom sequentially includes a first compartment 110, a second compartment 111 and a third compartment 112, the first compartment 110 accommodates a circuit board of the actuation module 14, the second compartment 111 accommodates a battery 130 and a metal electrode sheet 131 of the energy module 13, the third compartment 112 accommodates a circuit board of the control module 15, and the circuit board of the actuation module 14 and the circuit board of the control module 15 are connected through an antenna 150 of the control module 15, the antenna 150 is wrapped onto a sidewall of the assembly frame 11. Another hollow sidewall of the assembly frame 11 having the antenna 150 is provided for assembling the assembly part 12, and the top side of the assembly frame 11 correspondingly assembled with the assembly part 12 is concavely formed with an assembly slot 113, and the front end of the assembly slot 113 is provided with a second concave arc 1130.

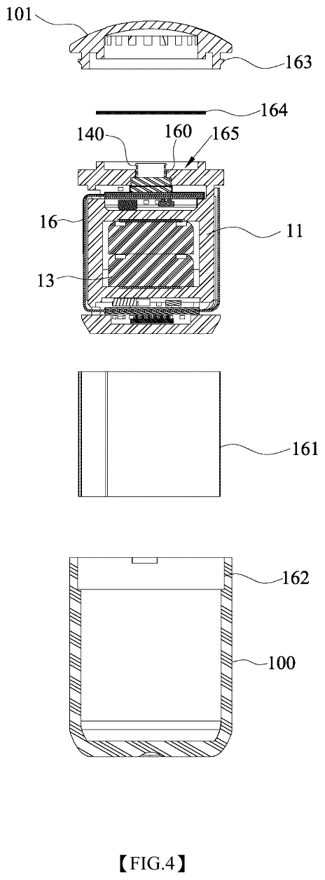

The hollow sidewall structure of the assembly part 12 corresponding to the assembly frame 11 is provided with a first part 120 which is an arc sheet, the left and right sides of the first part 120 extend in an arc to form a second part 121 and a third part 122 respectively, the area of the second part 121 and the area of the third part 122 are smaller than the area of the first part 120, such that the assembly part 12 is substantially cross shaped. The upper side of the first part 120 corresponding to the assembly slot 113 extends vertically forward to form a fourth part 123, and the front end of the fourth part 123 is configured to be corresponsive to the second concave arc 1130 and provided with a first concave arc 1230. While the assembly part 12 is assembled to the assembly frame 11, the fourth part 123 assembled to the assembly slot 113, and the first concave arc 1230 is connected to the second concave arc 1130 to form a round hole for accommodating an actuating element 140 of the actuation module 14. Therefore, the energy module 13, the actuation module 14 and the control module 15 can be securely installed between the assembly frame 11 and the assembly part 12 to produce a semi-assembled product to be placed in the seal structure. In this embodiment, part or all of the circuit components of the actuation module 14 and the control module 15 are coated with an insulating material. For an example as shown in FIG. 4, except the insulating material is not coated onto the top of the actuating element 140, which is exposed to the human body, the surface of the circuit board of the actuation module 14, or even part or all of the circuit board of the control module 15 and the antenna are coated with the insulating material such as a non-biocompatible conformal coating to form an insulating layer 16, so as to improve the overall structural insulation and perfect the shelf life and product quality. The actuating element 140 can be a pressure sensor, a temperature sensor or a pH sensor, where the pressure sensor can be a piezoelectric pressure sensor, a piezoresistive pressure sensor, a capacitive pressure sensor, an electromagnetic pressure sensor or a vibrating wire pressure sensor. The piezoelectric pressure sensor converts the measured pressure into electric energy by the piezoelectric effect, and it is made of a piezoelectric material such as sodium dihydrogen phosphate, sodium potassium tartrate or quartz, etc. and while an external force is acted on the piezoelectric material, electric charges are formed on the surface of the piezoelectric material, and after the charges are amplified, the sensed pressure is converted into a proportional electrical output.

The piezoresistive pressure sensor uses the piezoresistive effect to change the resistance of a material under stress. Unlike the piezoelectric effect, the piezoresistive effect only produces impedance changes and does not generate charges. The piezoresistive effect in semiconductor materials is much greater than that in metals. Silicon can be used as the main material. The piezoresistive pressure sensor is generally connected to a Wheatstone bridge via leads. While no pressure is applied to the sensitive core, the electric bridge is in a balanced state. While the sensor is under pressure, the chip resistance changes and the electric bridge loses balance. A constant current or constant voltage power source is added to the electric bridge. The electric bridge will output a voltage signal corresponding to the pressure and use the resistance change of the sensor to convert the voltage signal into a pressure signal output by the electric bridge. Therefore, the change in the resistance value detected by the electric bridge is amplified, and then converted into a corresponding current signal through voltage-current conversion.

The working principle of the electromagnetic pressure sensor including the inductive pressure sensor, the Hall pressure sensor and the eddy current pressure sensor is described below. The inductive pressure sensor is made of various magnetic materials with different magnetic permeabilities. While pressure acts on the membrane of the sensor, the size of the air gap changes. The change in the air gap affects the change in the inductance of the coil and converts the change in inductance into a corresponding signal output to achieve the purpose of measuring pressure. The Hall pressure sensor is made of semiconductor materials based on the Hall effect. The Hall effect refers to placing a solid conductor in a magnetic field and passing a current through the solid conductor. Under the action of the Lorentz force, the current in the conductor deflects to the a side and generates a voltage (Hall voltage). The electric field force caused by the voltage will balance the Lorentz force. Through the polarity of the Hall voltage, it can be confirmed that the current inside the conductor is caused by the movement of negatively charged particles (free electrons). The eddy pressure sensor is a pressure sensor with an eddy current effect, which is caused by the intersection of moving magnetic field and metal conductor, or caused by the perpendicular intersection of moving metal conductor and magnetic field.

The capacitive pressure sensor is a pressure sensor that converts the measured pressure into a change in capacitance, and it can use a round metal film or metal plating as the electrode of the capacitor. While the film is deformed under pressure, the capacitance formed between the film and the fixed electrode changes. The electrical signal output by the measuring circuit has a corresponding relationship with the voltage. The vibrating wire pressure sensor is a sensor that uses time and frequency signals to sense pressure.

The temperature sensor is a contact temperature sensor, which can be a thermistor or a resistance thermometer. The pH value sensor can be a sensor made of a polymer material or a special metal coated by a micro-electromechanical process, and the reaction of such special metal is used to detect the pH value.

The energy module 13 is electrically connected to the actuation module 14 and the control module 15, the actuation module 14 is telecommunicatively connected to the control module 15, and the control module 15 is telecommunicatively connected to the monitoring system through the antenna 150. The control module 15 includes a contactless switch such as a magnetic switch or a radio frequency switch, and while the capsule sensor structure 1 is put into a living organism and the contactless switch is turned on, the control module 15 receives an instruction of the monitoring system to drive the actuation module 14 to perform biosignal detection or physical therapy and give a feedback of an operation result, and the control module 15 transmits the operation result to the monitoring system for remote monitoring.

In order to prevent the shelf life of the structure from being reduced due to the spillage or leakage of the adhesive material during the dispensing process while the capsule sensor structure 1 is dispensed with an adhesive and assembled, after the actuating element 140 is received by the round hole, the periphery of the round hole is dispensed with an adhesive to form a first sealing ring 160 and assemble the gap in an airtight manner, and then the assembly track of the assembly part 12 and the assembly frame 11 is dispensed with an adhesive to assemble the gap in the airtight manner, so as to perfect the waterproof property of the overall structure, or the surfaces of the assembly frame 11 and the assembly part 12 are dispensed with a biocompatible epoxy resin after they are assembled with each other is assembled with the assembly part 12 to further improve the structural insulation; the peripheries of the assembly part 12 and the assembly frame 11 assembled with each other, that is, the outer periphery of the semi-assembled product is sheathed with a solder mask tape 161 to further enhance the insulation protection of the main circuit components in the structure, so as to prevent the components from being damaged due to the leakage of current caused by accidentally spilling the adhesive materials during the dispensing and assembly processes.

An open end of the accommodation slot 100 is provided with a glue overflow groove 162 for avoiding the current leakage issue caused by excessive adhesive materials while the semi-assembled product is put into the seal structure and assembled with the accommodation slot 100 and the cap 101. The capsule sensor structure 1 further includes an O-ring 163 installed to the cap 101 and clamped between the cap 101 and the accommodation slot 100 to improve the sealing strength between the cap 101 and the accommodation slot 100, so as to effectively prevent the penetration of biological tissue fluid or water moisture.

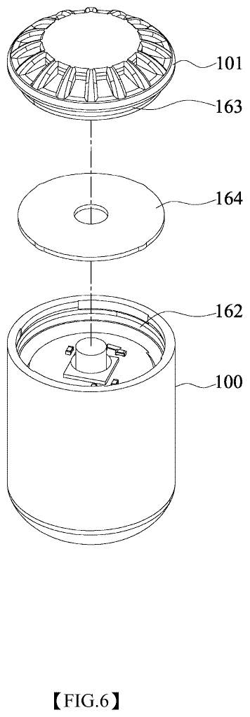

Since the cap 101 has a plurality of filter holes and a cover for filtering impurities, resisting water, and providing a breathable effect in order to allow the actuating element 140 in the living organism to be free from being distributed by biological tissue fluids, therefore the capsule sensor structure 1 further includes a filter membrane 164 to ensure that the normal operation of the actuating element 140 and the waterproof breathable effect of room included between the actuating element 140 and the cap 101, and the filter membrane 164 is clamped between the assembly part 12 and the assembly frame 11, or clamped between the cap 101 and the assembly frame 11 assembled with the assembly part 12, and covered onto the actuation module 14 to form another layer of waterproof breathable protective membrane, or the filter membrane 164 is attached to the top of the assembly frame 11 dispensed with an adhesive and assembled with the assembly part 12. In order to prevent the adhesive materials from overflowing or spilling to contaminate the actuation module 14, the top of the assembly frame 11 assembled with the assembly part 12 is concavely provided with at least one dispensing groove 165 for receiving the adhesive materials and dispensing and attaching the filter membrane 164 to the assembly frame 11. It is noteworthy that the filter membrane 164 may be in the shape of a donut as shown in FIG. 6, and an opening is formed at the center of a round slice for accommodating the actuating element 140, and the filter membrane 164 is attached onto the actuation module 14, and the periphery of filter membrane 164 is connected to the opening at the top of the accommodation slot 100; the periphery of the opening is dispensed with an adhesive to form a second sealing ring, so as to improve the waterproof property, and the donut shaped filter membrane 164 and the opening of the accommodation slot 100 can be dispensed with an adhesive to improve the sealing strength and perfect the structural waterproof property of the overall structure, so as to provide a highly waterproof capsule sensor structure.

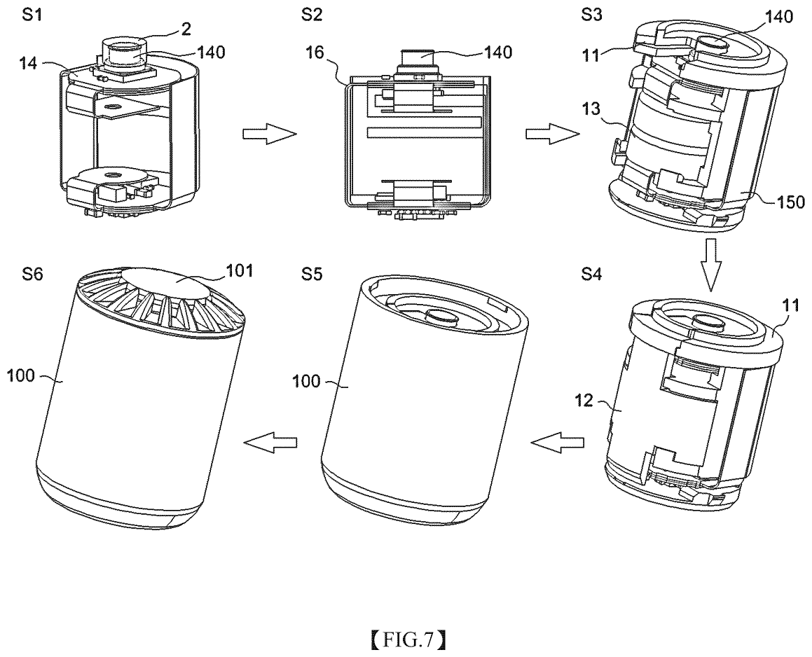

With reference to FIG. 7 for the schematic diagram showing the assembly procedure of a capsule sensor structure of a third preferred embodiment of the present disclosure, the assembly method of the capsule sensor structure 1 includes the following steps S1-S6. Step S1: Cover a silicone protective cover 2 onto the actuating element 140 of the actuation module 14; Step S2: Completely immerse the circuit board of the actuation module 14 into the insulating material (not shown in the figure), and set it still in the insulating material to solidify to form the insulating layer 16 that covers the surface of the circuit board of the actuation module 14, and then remove the silicone protective cover 2; Step S3: Assemble the circuit board of the actuation module 14 to the upper part of the assembly frame, and assemble the circuit board of the control module 15 to the lower part of the assembly frame 11, wrap the antenna 150 around a sidewall of the assembly frame 11, and then assemble the energy module 13 into the assembly frame 11; Step S4: Assemble the assembly part 12 and the assembly frame 11 to form the semi-assembled product; Step S5: Put the semi-assembled product into the accommodation slot 100; and Step S6: Dispense and assemble the cap 101 and the accommodation slot 100. It is noteworthy that the housing can be made of a polycarbonate (PC) containing biocompatible material which is convenient for the use by living organisms, and the assembly frame and the assembly part can be made of a polyamide (PA) containing non-biocompatible material for ensuring the resistance for the occlusal force occurred in the internal frame structure and enhancing the structural rigidity. The assembly frame 11 and the assembly part 12 are made of polyamide (PA) and a hardness strengthening material, such as a fiber material, and the fiber material can be fiberglass or carbon fiber which accounts for 10-40% of the total production materials 10-40% and perfects the supporting structure of the frame structure.

What is claimed is:

1. A capsule sensor structure, telecommunicatively coupled to an external monitoring system, comprising:
a housing, substantially in the shape of a capsule body, and having an accommodation slot and a cap, the accommodation slot being dispensed with an adhesive and bonded with the cap to form a seal structure;
an assembly frame, installed in the seal structure;
an energy module, installed in the seal structure;
an actuation module, installed to a side of the assembly frame and electrically coupled to the energy module; and
a control module, installed to a side of the assembly frame and electrically coupled to the energy module and telecommunicatively coupled to the actuation module, and the control module having an antenna for telecommunicatively coupling the monitoring system;
wherein, while the capsule sensor structure is put into a living organism, the control module receives an instruction of the monitoring system to drive the actuation module to perform a biosignal detection or a physical therapy and to feed back an operation result, and the control module transmits the operation result to the monitoring system for remote monitoring.

2. The capsule sensor structure according to claim 1, further comprising an assembly part provided for being assembled and coupled to the assembly frame to securely install the energy module, the actuation module and the control module in the assembly frame, the assembly frame being in a substantially cylindrical shape and from top to bottom sequentially comprising a first compartment, a second compartment and a third compartment, the first compartment accommodating a circuit board of the actuation module, the second compartment accommodating a battery and a metal electrode sheet of the energy module, the third compartment accommodating a circuit board of the control module; the antenna being wrapped onto a sidewall of the assembly frame, and another hollow sidewall of the antenna installed in the assembly frame being provided for placing the assembly part into the seal structure after assembly.

3. The capsule sensor structure according to claim 2, wherein the assembly part comprises a first part, the left and right sides of the first part extend in an arc forward to form a second part and a third part respectively, and both the area of the second part and the area of the third part are smaller than the area of the first part, the upper side of the first part extends vertically forward to form a fourth part, and the front end of the fourth part is provided with a first concave arc; the top side of the assembly frame correspondingly assembled with the assembly part is configured to be corresponsive to the fourth part and concavely formed with an assembly slot, and the front end of the assembly slot is configured to be corresponsive to the first concave arc and provided with a second concave arc, such that while the assembly part is assembled to the assembly frame and the fourth part is assembled to the assembly slot, the first concave arc is coupled to the second concave arc to form a round hole for accommodating an actuating element of the actuation module.

4. The capsule sensor structure according to claim 3, wherein the actuation module and the control module have part or all of the circuit components coated with an insulating material to form an insulating layer to improve the overall structural insulation.

5. The capsule sensor structure according to claim 4, wherein after the round hole accommodates the actuating element, the periphery of the round hole is dispensed with an adhesive to form a first sealing ring to improve the waterproof property.

6. The capsule sensor structure according to claim 5, wherein the inner side of the open end of the accommodation slot is surrounded with a glue overflow groove for accommodating excessive adhesive materials after the accommodation slot and the cap are dispensed with an adhesive and bonded with each other.

7. The capsule sensor structure according to claim 6, further comprising an O-ring installed to the cap and clamped between the cap and the accommodation slot to improve the sealing strength between the cap and the accommodation slot to further perfect the structural waterproof property.

8. The capsule sensor structure according to claim 7, wherein the control module comprises a contactless switch.

9. The capsule sensor structure according to claim 7, further comprising a filter membrane wrapped on the actuation module to prevent the actuating element from being damaged by the penetration of a biological tissue fluid or moisture.

10. The capsule sensor structure according to claim 9, wherein the filter membrane and an opening of the accommodation slot are dispensed with an adhesive and bonded with each other to improve the structural assembly sealing strength and perfect the structural waterproof property.

11. The capsule sensor structure according to claim 10, wherein the top side of the assembly frame assembled with the assembly part is concavely formed with at least one dispensing groove for preventing the adhesive material from overflowing and contaminating the actuation module while dispensing and adhering the filter membrane to the top side of the assembly frame assembled with the assembly part.

12. The capsule sensor structure according to claim 11, wherein the filter membrane is substantially in a donut shape, an opening is formed at the center of a round slice and provided for accommodating the actuating element, and the filter membrane is attached onto the actuation module and the periphery of the filter membrane is coupled to the opening above the accommodation slot; the periphery of the opening is dispensed with an adhesive to form a second sealing ring to improve the waterproof property.

13. The capsule sensor structure according to claim 2, wherein the housing is formed by a polycarbonate (PC) containing biocompatible material, and the assembly frame and the assembly part are made of a polyamide (PA) containing non-biocompatible material.

14. The capsule sensor structure according to claim 2, wherein the assembly frame and the assembly part is made of polyamide (PA) and a hardness strengthening material.

15. The capsule sensor structure according to claim 14, wherein the hardness strengthening material comprises a fiber material which accounts for 10-40% of the total production materials.

16. A method of assembling the capsule sensor structure of claim 1, comprising the steps of:
covering a silicone protective cover onto an actuating element of the actuation module;
completely immersing the circuit board of the actuation module in an insulating material, and setting it still in the adhering insulating material to solidify to form an insulating layer that covers the surface of the circuit board of the actuation module, and then removing the silicone protective cover;
assembling the circuit board of the actuation module to the upper part of the assembly frame, assembling the circuit board of the control module to the lower part of the assembly frame, surrounding the antenna around a sidewall of the assembly frame, and assembling the energy module into the assembly frame;
assembling the assembly part to the assembly frame to form a semi-assembled product;
placing the semi-assembled product into the accommodation slot; and
dispensing and assembling the cap and the accommodation slot.
